SEQUENCE LISTING (15 sequences)

SEQ ID NO: 1 - DNA, 27 bp, artificial sequence
  PCR primer
garytnaarctynggnttyatngcnca

SEQ ID NO: 2 - DNA, 28 bp, artificial sequence
  PCR Primer
ggcatnacnarrttncgnarnccnatna

SEQ ID NO: 3 - DNA, 30 bp, artificial sequence
  PCR Primer
cctctccaccatgcttgccaacctcttctc

SEQ ID NO: 4 - DNA, 30 bp, artificial sequence
  PCR Primer
ggagaagaggttggcaagcatggtggagag

SEQ ID NO: 5 - DNA, 30 bp, Artificial sequence
  PCR Primer
cggcggaggcagcagtcttagaatgagtag

SEQ ID NO: 6 - DNA, 29 bp, artificial sequence
  PCR Primer
ggggggactacaggggaaaacagggaaag

SEQ ID NO: 7 - DNA, 50 bp, artificial sequence
  PCR Primer
caccatgcttgccaacctcttctccatgaggtcattcataaagattgagg

SEQ ID NO: 8 - DNA, 50 bp, artificial sequence
  PCR Primer
cctcaatctttatgaatgacctcatggagaagaggttggcaagcatggtg

SEQ ID NO: 9 - DNA, 26 bp, artificial sequence
  PCR Primer
gttgggaagaacattgttctttgtgg

SEQ ID NO: 10 - DNA, 44 bp, artificial sequence
  PCR Primer
atttaggtgacactatagaagtggactttgacagagaaagtttg

SEQ ID NO: 11 - DNA, 23 bp, artificial sequence
  PCR Primer
atttaggtgacactatagaagtg

SEQ ID NO: 12 - DNA, 3773 bp, Homo sapiens
cggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcgttgggaagaacattgttctttgtggagactggaaagcaagggaaatgtgagaagattaaactactgcaggggtcagcaaactttctctgtcaaagtcaaggttgcagctagatcacgctattccaaagatccatttgagttcaagaaggagactcccaattctcggcttgtgaccgagccagttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatccgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtcccccc

SEQ ID NO: 13 - DNA, 99 bp, Homo sapiens
cgttgggaagaacattgttctttgtggagactggaaagcaagggaaatgtgagaagattaaactactgcaggggtcagcaaactttctctgtcaaagtc

SEQ ID NO: 14 - protein, 1174 aa, Homo sapiens
MDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRRWEEHCSLWRLESKGNVRRLNYCRGQQTFSVKVKVAARSRYSKDPFEFKKETPNSRLVTEPVEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDPFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 15 - protein, 33 aa, Homo sapiens
RWEEHCSLWRLESKGNVRRLNYCRGQQTFSVKV

(19) United States
(12) Patent Application Publication
(10) Pub. No.: US 2004/0081973 A1
(43) Pub. Date: Apr. 29, 2004

(54) Cloning and characterization of a novel BK channel isoform highly expressed in glioma cells

(76) Inventors: Harald W. Sontheimer, (US); Xiaojin Liu, Boston, MA (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/375,293

(22) Filed: Feb. 27, 2003

Related U.S. Application Data
(60) Provisional application No. 60/360,384, filed on Feb. 28, 2002.

(51) Int. Cl. C12Q 1/68; C07H 21/04; C07K 14/47; C12P 21/02; C12N 5/06

(52) U.S. Cl. 435/6; 435/69.1; 435/320.1; 435/325; 530/350; 536/23.5

(57) ABSTRACT

Described is the cloning and functional characterization of a novel splice variant of hSlo, the gene encoding the α-subunit of IbTX sensitive human BK channels. The novel isoform of BK channels, which was termed gBK, contains a 63 amino acid insert at splice site 2, which differs by 33 amino acids from its nearest relative hbr5. gBK channels were over-expressed in glioma cells as evident from examination of human biopsy specimens. Moreover, gBK channel expression correlated positively with the relative degrees of malignancy of the tumor tissues. Heterologous expression of gBK in oocytes revealed that the pharmacological and biophysical properties of gBK are consistent with the properties of native BK currents in glioma cells. Furthermore, even when compared with its most homologous form hbr5, gBK showed distinct properties, including slowed channel activation and importantly, enhanced Ca²⁺ sensitivity at physiologically relevant [Ca²⁺]ᵢ values.

### FIELD OF DISCLOSURE

This application claims the benefit of U.S. Provisional Patent Application No. 60/360,384, filed Feb. 28, 2003. The present disclosure relates generally to K⁺ channels. Specifically, the present disclosure relates to the identification and characterization of a novel BK channel from human glial tissue.

### BACKGROUND

There are a variety of K⁺ channels subtypes involved in human physiology. These K⁺ channels regulate a myriad of processes, ranging from regulation of the heartbeat to control of the excitability of nerve cells. Several classes of K⁺ channels have been described based on their pharmacological properties and electrophysiological properties, and include the voltage gated K⁺ channels, the ATP-regulated K⁺ channels, Ca⁺-activated K⁺ channels, Na⁺-activated K⁺ channels, and K⁺ channels activated in response to specific compounds. An important member of the K⁺ channel family includes the voltage-dependent large-conductance Ca²⁺-activated K⁺ channels (BK channels). Intracellular calcium concentration [Ca²⁺]i and membrane potential regulate the BK channels. As an example, BK channels are opened to allow K⁺ efflux in response to an increase in intracellular Ca²⁺ concentration. Therefore, modulation of BK channel activity impacts various pathways that depend on an influx of calcium through voltage dependent pathways.

The BK channels are widely expressed in excitable and nonexcitable cells. BK channels can be composed of either an α-subunit homodimer, or a heterodimer comprising an α-subunit and a β-subunit. BK channels from most mammalian neurons are not thought to be associated with a β-subunit. BK channels exhibit diverse electrophysiological properties, which are in part due to alternative splicing of their α-subunits. Regulation by the β-subunit may also play a role in BK channel function. BK channels, resemble a unique class of ion channels that couple intracellular chemical signaling to electric signaling (McManus, 1991). BK currents have been implicated in growth control of glial cells and BK channels with novel biophysical properties have been characterized in human glioma cells. BK channels have been indicated to regulate neuronal firing (MacDermott and Weight, 1982; Robitaille and Charlton, 1992; Robitaille et al., 1993; Poolos and Johnston, 1999; Golding et al., 1999), endocrine cell secretion (Marty, 1989; Lingle et al., 1996), and smooth muscle tone (Nelson and Quayle, 1995; Brenner et al., 2000). In nonexcitable cells, such as epithelial, endothelial, or glial cells, BK channels may contribute to diverse biological functions ranging from osmoregulation (Turnheim et al., 1989), cell proliferation (Wiecha et al., 1998), to cell migration (Soroceanu et al., 1999).

This remarkable functional diversity of BK channels may, in part, be explained by the molecular diversity of their pore-forming α subunits. These α subunits derive from a single gene (Slo) that undergoes extensive alternative splicing. It has been demonstrated that alternative splicing of the Slo gene leads to BK channels with different biophysical properties, and these diverse channels may serve different biological functions (Lagrutta et al., 1994; Xie and McCobb, 1998; Jones et al., 1999; Ramanathan et al., 1999). So far, five alternative splicing sites have been identified in hSlo (Tseng-Crank et al., 1994; Ferrer et al., 1996). Of these, splice site 2 appears to be the most heterogeneous not only in hSlo, but also in dSlo (Drosophia Slo) (Atkinson et al., 1991; Adelman et al., 1992), mSlo (mouse Slo) (Butler et al., 1993), and rSlo (rat Slo) (Xie and McCobb, 1998). Most notably, changes at splice site 2 appear to affect the Ca²⁺ sensitivity of the channel. For example, in rat adrenal chromaffin cells and PC12 cells, the presence of a cysteine-rich 59 amino acid exon (STREX-1) increased the apparent Ca²⁺ sensitivity of BK channels when compared with the channels without the exon (ZERO) (Saito et al., 1997; Hanaoka et al., 1999). Similarly, in chick cochlea, a 61 amino acid exon at this site enhances the Ca²⁺ sensitivity of the channel (Ramanathan et al., 1999). In addition to alterations in Ca²⁺ sensitivity, there is evidence that exons at splice site 2 can alter the regulation of BK channels by kinases involved in intracellular signaling events.

BK channels have been studied in astrocytes (Nowak et al., 1987), human gliomas (Zahradnikova and Zahradnik, 1992) and Muller glial cells, and have been suggested to participate in the proliferation of nonexcitable cells, including glial cells. For example, BK channels have been implicated in the regulation of cultured Muller cell (specialized retinal glial cells) proliferation (Puro et al., 1989; Kodal et al., 2000; Newman, 1985; Bringmann and Reichenbach, 1997; Bringmann et al., 1997). In Muller cells, the enhanced activity of BK channels correlated with the increase of cell proliferation during development and following injury (gliosis) (Bringmann et al., 2000). Interestingly, current amplitudes of BK channels are significantly larger in Muller cells isolated from patients with proliferative vitreoretinopathy (PVR) than in cells from healthy donors (Bringmann et al., 1999) suggesting a possible role for BK channels in this proliferative disease.

Glioma cells are transformed glial cells that have lost their growth control. The process of the glial to glioma transition is poorly understood. Over-expression of growth factor receptors, most notably the EGF receptor (EGF-R), is a characteristic feature of glioma cells (Collins, 1994), and its activation has been shown to increase [Ca²⁺]ᵢ (Hernandez et al., 2000). Increase in intracellular Ca²⁺ is required during G1/S transition of the cell cycle (Whitfield et al., 1995).

BK channel expression has also been linked to oncogenic cell transformation. For example, Huang (Huang and Rane, 1994) reported that p21^{ras}, which plays a pivotal role in controlling cell oncogenic transformation (Ding et al., 2001), and its immediate downstream target, the Raf kinase, are required for the induction of Ca²⁺-activated K⁺ channels (Kca). Increased activity of Kca channels appeared to be required for the mitogenic stimulation of non-transformed cells with EGF and PDGF. Mitogenic stimulation in the presence of the Kca blocker charybdotoxin (ChTX) inhibited the stimulatory effect of the mitogen, suggesting that Kca is one of the physiological targets of p21^{ras} and may play a role in cell proliferation (Huang and Rane, 1994). The transformation of CEFs with Rous sarcoma virus (RSV) specifically induced a TEA (IC₅₀=1.8 mM) and ChTX (IC₅₀=19 nM)-sensitive Kca current that is absent from nontransformed cells (Repp et al., 1993).

In addition to roles in cell proliferation and oncogenic transformation, BK channels have also been linked to cell migration and tumor cell invasion. Glioma cells show an unusual ability to diffusely invade the normal brain thereby escaping surgical treatment (Merzak and Pilkington, 1997). It has been suggested that cell invasion into narrow brain spaces may require tumor cells to shrink. Cell shrinkage requires efflux of KCl and BK channels may serve as the pathway for regulated K⁺ efflux (Christensen and Zeuthen, 1987; Gitter et al., 1987). Consistent with this notion, 10 nM iberiotoxin (IbTX) was found to reduce the in vitro invasion of U251-MG glioma cells (Soroceanu et al., 1999). In addition, the BK channel specific blocker IbTX has been shown to inhibit migration of U-251MG glioma cells in vitro (Soroceanu et al., 1999). Furthermore, two human glioma cell lines (STTG-1, WHO Grade III, and D-54MG, WHO Grade IV) exhibit large BK currents that are more sensitive to [Ca²⁺]ᵢ (Ransom and Sontheimer, 2001) than has previously been reported for other types of BK channels (Tseng-Crank et al., 1994; DeCoursey et al., 1996; Hurley et al., 1999).

Although BK channels have been linked to the regulation of various intracellular processes, including, but not limited to, cell proliferation, cell migration, oncogenic transformation and tumor cell invasion, the function and the identity of BK channels in glial and glioma cellular physiology is not clearly understood. The present disclosure describes the cloning, isolation and characterization of a novel BK channel from human glioma which contains a unique hSlo splice variant at splice site 2 of the BK channel. This alternative splice variant of hSlo encodes a channel that contains a unique 33 amino acid exon at splicing site 2 of hSlo and is referred to herein as glioma BK (gBK).

### SUMMARY

The present disclosure is directed to a novel isolated and purified nucleic acid molecule which encodes the α-subunit of gBK, or a functional derivative thereof. It is further directed to an expression vector for expression of the novel gBK channel in a recombinant host, wherein said vector contains a recombinant nucleic acid encoding the gBK channel, or functional derivative thereof, along with the genetic elements required for expression (expression control sequence) as defined herein. Further, this disclosure is directed to a recombinant host cell containing a recombinantly cloned nucleic acid encoding the novel gBK channel, or functional derivative thereof. The present disclosure is also directed to a process for expression of the novel gBK channel polypeptide in a recombinant host cell, comprising: (1) transferring the expression vector containing a recombinant gene encoding the gBK channel, or functional derivative thereof, into suitable host cells; and (2) culturing the host cells of step (1) under conditions which allow expression of gBK from the expression vector.

In addition, the instant disclosure is directed to a polypeptide encoded by the gBK nucleic acid sequence, or a derivative thereof. The disclosure is also directed to a antibodies immunologically reactive with the gBK channel polypeptide, or a functional derivative thereof.

Further, this disclosure is directed to a method of identifying compounds that modulate the novel gBK channel activity, comprising: (1) combining a suspected gBK channel modulator and the gBK channel (or a functional derivative thereof), the gBK channel being either alone, or in combination with a modulating compound, in an appropriate assay system; and (2) measuring an effect of the modulator on the activity of the gBK channel, or functional derivative thereof. The modulator may be either an agonist or antagonist.

This disclosure is also directed to a compound active in the aforementioned method, wherein the compound is a modulator of the gBK channel, or a functional derivative thereof, alone, or a modulator of the novel gBK channel in combination with a β-subunit of a mammalian calcium-activated potassium channel, or a functional derivative thereof. Further, this disclosure is directed to a pharmaceutical composition comprising a compound active in the aforementioned method.

### BREIF DESCRIPTION OF THE DRAWINGS

FIG. 1 illustrates analysis of BK channel expression in glioma and non-glioma tissues. The polyclonal BK antibody MP-2 was used to examine the expression of BK channels by Western blot in human biopsy tissue, with autopsy samples from normal human brain serving as control. Antibodies consistently detected a BK channel protein band at a molecular weight of ˜120 kDa. This band was significantly enhanced in glioma samples. A band at ˜42 kDa corresponds to α-actin, which was used as a loading control. Human biopsy tissue samples from glioma patients showing enhanced BK channel expression. Tissue samples were grouped into five categories, from left to right: C. normal cortex from autopsy, I. Pilocytic astrocytoma (WHO, Grade I), II. Grade II Astrocytoma (WHO, Grade II), III. Anaplastic astrocytoma (WHO, Grade III), IV. Glioblastoma (WHO, Grade IV).

FIG. 2 shows analysis of BK channel expression in cultured glioma cell lines. The polyclonal anti-BK antibody MP-2 was used to examine the expression of BK channels by Western blot in established human glioma cell lines. The first three lanes show normal human cortex samples (control). The three glioma cell lines studied were STTG-1, derived from a WHO grade III astrocytoma, D54-MG and U251-MG each derived from WHO grade IV glioblastoma multiforme.

FIG. 3 illustrates the gBK channel is a novel, alternatively spliced, BK channel isoform derived from the hSlo gene. A schematic drawing of a BK channel with the five identified splicing sites is illustrated. The transmembrane α helices S0 to S6 and the C-terminal hydrophobic domains S7 to S10 are represented by gray barrels. Amino acid sequences of the alternate splice exons of gBK and hbr5 are illustrated. The underlined sequence represents a 29 amino acid exon that is present in both gBK and hbr5. The sequence in bold represents the novel exon of gBK. The symbols above the sequence point to motifs that meet the criteria for: *. Casein kinase I phosphorylation site, ♦, multifunctional calmodulin dependent kinase phosphorylation site, and ●, protein kinase C phosphorylation site.

FIG. 4 illustrates the distribution and relative expression levels of gBK channels in normal and neoplastic human tissues. Normalized cDNAs were used to detect the novel insert in eight normal human tissues and eight neoplastic human samples representing tumors derived from six different organs. The primers (SEQ ID NOS. 9 and 10) were designed specifically to amplify the gBK insert. Glioma cDNA library was used as positive control. To ensure the specificity of the primers, PCR product from glioma cDNA library was sequenced with sp6 primer (SEQ ID NO. 11). The expression level of the house-keeping gene G3PDH was used as internal control. The top panel shows amplification results with the gBK insert specific primers, all tissues examined here show a ˜120 bp product, a size that is consistent with what was obtained from the glioma cDNA library. The bottom panel shows results with human G3PDH specific primers, which give rise to a 983 bp amplification product.

FIG. 5 illustrates western blot analysis of gBK and hbr5 injected Xenopus oocytes showing BK channel protein can be expressed in oocytes. Uninjected oocyte lysates (control) was used as negative control and hbr5 injected oocyte lysates (hbr5) was used as positive control. The anti-BK channel antibody MP-2 recognized a specific band of approximately 120 kDa in both gBK and hbr5 injected oocyte lysates, but not in uninjected control oocyte lysate.

FIG. 6 illustrates gBK forms functional, IbTX-sensitive BK channels in Xenopus oocytes. gBK injected oocytes express large voltage-activated outward currents. Current recordings were obtained by double-electrode voltage-clamp in OR2 medium containing 92.5 mM NaCl, 2.5 mM KCl, 1 mM CaCl₂, 1 mM MgCl₂ and 5 mM HEPES, pH 7.5, with 3 M KCl containing microelectrodes. Oocytes were held at −20 mV and stepped to test potentials between 0 and +180 in 20 mV increments. Uninjected oocytes were used as control and currents shown are averages currents from 3 oocytes (left panel). gBK injected oocytes showed large voltage-dependent outward currents, traces shown are averages from 5 oocytes (right panel). To confirm that the outward currents were mediated by BK channels, the highly selective BK channel blocker IbTX was applied. Greater than 60% of total currents were inhibited by 100 nM IbTX.

FIG. 7 illustrates the dose-response of IbTX inhibition of gBK currents in gBK injected oocytes. The left panel shows the normalized current (I/Iₘₐₓ)-voltage relationship for total currents before IbTX treatment (open circles), the residual currents after 225 nM IbTX treatment (open triangles), and the IbTX-sensitive currents (closed circles) that were obtained by subtracting residual currents from total currents. The right panel shows pooled IbTX dose-responses from three oocytes (three symbols represent data from three oocytes respectively) that were obtained by plotting the relative inhibition achieved by IbTX as a function of the applied IbTX concentration. The data were least-squares fitted to a modified Hill equation of the form: I/Iₘₐₓ=1/(1+([IbTX]/IC₅₀)ⁿ), where I is the IbTX sensitive current, Iₘₐₓ is the maximum current of the fit, IC₅₀ is the half-maximal inhibitory concentration of IbTX, and n is the Hill coefficient. The dose-responses were obtained after applying IbTX up to 20 min until it reached steady states and the continuous line was obtained from average data of three oocytes.

FIG. 8 illustrates single-channel recordings from gBK expressed in Xenopus oocytes.

FIG. 8A shows representative single-channel recordings for gBK. Single-channel currents were measured in inside-out patches at different holding potentials under 100 nM free [Ca²⁺]ᵢ in 145 mM symmetrical K-gluconate. The closed states of the channels are indicated by dashed lines. Data were filtered at 2 kHz and digitized at 10 kHz.

FIG. 8B shows the single-channel I-V relation for gBK (closed circles). Current amplitudes were obtained by Gaussian fit of amplitude histograms. The resulting I-V relation was fitted using linear regression and the single channel conductance was obtained from the slope of the fitted line. Data are expressed as mean±S.D.

FIG. 9 illustrates differential activation kinetics of gBK and hbr5 currents. Currents were studied in macropatches from oocytes injected with either gBK or hbr5. For comparison, current traces elicited at each test voltage were normalized to the maximum amplitude and traces from each patch were averaged from four consecutive recordings. The thick line represents gBK mediated currents, the thin line represents hbr5 mediated currents. The patches were held at 0 mV and depolarized to the testing voltages for 200 ms. Currents were recorded in 145 mM symmetrical K-gluconate solutions with different free [Ca²⁺]ᵢ.

FIG. 9A shows averages of normalized traces recorded during superfusion with internal solution buffered to 100 nM free [Ca²⁺]ᵢ from seven patches of gBK and six patches of hbr5 injected oocytes at +60 mV and +80 mV testing voltages, respectively.

FIG. 9B shows the relations between fast rise time constants (Tau_{fast}) and voltage for both gBK (open circles) and hbr5 (closed circles) mediated currents at 100 nM free Ca²⁺. The asterisks identify rise time constant values that differ significantly between gBK and hbr5 at the same voltage (p<0.05). The error bars indicate±SEM.

FIG. 9C shows averages of normalized currents recorded at 1 μM free Ca²⁺ from three patches of gBK and five patches of hbr5 mRNA injected oocytes, respectively.

FIG. 10 illustrates the calcium sensitivity of gBK and hbr5. Currents were elicited with 200 ms voltage steps to test potentials between −120 and +160 mV in 20-mV increments from a 0 mV holding potential in inside-out patches and recorded during superfusion with internal solutions buffered to different free [Ca²⁺]ᵢ ranging from 0 and 1 μM. Data are expressed as means±SEM.

FIG. 10A shows calcium dependence of gBK. Normalized conductances (G/Gₘₐₓ) of gBK (A, n=5) at +80 mV testing potential were plotted against free [Ca²⁺]ᵢ (see materials and methods) and fitted to the Hill equation: G=Gₘₐₓ/(1+(K_{d}/[Ca]ᵢ)ⁿ, where Gₘₐₓ is the maximum conductance of the fit, K_{d} is apparent Ca²⁺ dissociation constant, n is the Hill coefficient.

FIG. 10B shows calcium sensitivity of gBK. Normalized conductance (G/Gₘₐₓ) of gBK at five different Ca²⁺ concentrations were plotted against voltage and fitted to a Boltzmann equation: G/Gₘₐₓ=1/(1+exp(−q(V−V_{1/2})/kT)), where G/Gₘₐₓ is normalized conductance, q is the effective gating charge, V_{1/2} is the half-maximal voltage, k is the Boltzmann constant, and T is temperature in Kelvin.

FIG. 10C shows the V_{1/2}-[Ca²⁺]ᵢ Relations differ significantly (p<0.05) between gBK (n=6) and hbr5 (n=10).

FIG. 11 illustrates the Ca²⁺ dependence of TEA-sensitive currents in outside-out patches from human glioma cells.

FIG. 11A illustrates currents responses from three representative outside-out patches from STTG-1 and D54MG glioma cell lines at 0 M [Ca²⁺]ᵢ, 1.5×10−7 M [Ca²⁺]ᵢ and 2.1 ×10−6 M [Ca²⁺]ᵢ. The dashed line in the rightmost panel indicates a zero current level.

FIG. 11B shows activation curves (normalized conductance as a function of membrane potential) derived from the data in FIG. 11A. The V_{0.5} and apparent gating charges (q) are indicated. Data points are the mean +/−S.D. of normalized conductance from 5-20 voltage steps to different potentials for each patch.

FIG. 12 illustrates the Ca²⁺ dependence of TEA-sensitive currents in outside-out patches from human glioma cells.

FIG. 12A illustrates currents responses from three representative outside-out patches from HEK cells expressing hbr5 at 0 M [Ca²⁺]ᵢ, 2.1×10−6 M [Ca²⁺]ᵢ and 3.3×10−6 M (note the different voltage steps for the rightmost panel, and different scale bars as compared to FIG. 11). The dashed line in the rightmost panel indicates a zero current level.

FIG. 12B shows activation curves (normalized conductance as a function of membrane potential) derived from the data in FIG. 11A. The V_{0.5} and apparent gating charges (q) are indicated. Data points are the mean +/−S.D. of normalized conductance from 5-20 voltage steps to different potentials for each patch.

FIG. 13 illustrates tetrandrine sensitivity of gBK and hbr5 currents.

FIG. 13A shows gBK currents in an outside-out patch evoked with voltage steps to +120 mV with 3 or 30 uM tetrandrine. Displayed currents are the average of 5 voltage steps.

FIG. 13B shows hbr5 currents in an outside-out patch evoked with voltage steps to +120 mV with 3 or 30 uM tetrandrine. Displayed currents are the average of 5 voltage steps.

FIG. 13C shows a summary of tetrandrine inhibition of gBK and hbr5 currents at 3 and 30 uM.

### DETAILED DESCRIPTION

In the present disclosure, the isolation, identification, cloning and functional characterization of gBK, a novel splice variant of hSlo is described. The gBK polypeptide contains a 33-amino acid insert at splice site 2 in C-terminal tail of BK channels. gBK channels are ubiquitously expressed in various normal tissues as well as neoplastic samples, suggesting that the novel gBK channel may modulate important physiological functions. In addition, gBK channel expression correlates positively with the relative degrees of malignancy of the tumor tissues. As discussed above, BK channels have been implicated in cellular proliferation and neoplastic transformation. This disclosure is the first report demonstrating over expression of BK channels in gliomas acutely removed from patients. The positive correlation of gBK channel expression with tumor malignancy grade suggests a role of gBK channels in the biology of these tumors. Heterologous expression of gBK in oocytes revealed that the pharmacological and biophysical properties of gBK are consistent with the properties of native BK currents in glioma cells. This observation was confirmed in gBK channels isolated from glioma cell lines. Furthermore, even when compared with its closest homologue, hbr5, gBK showed distinct properties, including slowed channel activation and importantly, enhanced Ca²⁺ sensitivity at physiologically relevant [Ca²⁺]ᵢ values.

The novel exon at splice site 2 in gBK channels is the fifth exon described for splicing site 2 in BK channels, and splicing variants containing this exon are ubiquitously present among different tissues studied here. The physiological properties of BK channels containing variant exons at splice site 2 suggests that splice site 2 exons may either be a part of the Ca²⁺ sensor of BK channels, or may interact with a Ca²⁺ sensor located elsewhere on the BK channel (Tseng-Crank et al., 1994). The data described herein is consistent with this hypothesis and demonstrates that gBK has even greater Ca²⁺ sensitivity under physiological relevant [Ca²⁺]ᵢ than any previously described BK channel. In addition to alterations in Ca²⁺ sensitivity, there is evidence that exons at splice site 2 can alter the regulation of BK channels. The present disclosure shows that novel amino acid insert at splice site 2 of gBK contains at least three potential sites meeting the consensus criteria for kinase phosphorylation by casein kinase I, CAMK II and PKC, raising the possibility that the gBK channels may be regulated by intracellular kinases. In addition, this observation suggests that gBK channels may be subject to unique modulation by compounds that interact with intracellular kinases. Further, BK channels may be used to decipher the downstream signaling events mediated by gBK channels. If a protein is believed to be involved in gBK signaling events, this protein can be targeted for inhibition, and the effect of this inhibition on the phosphorylation state of gBK can be examined. Proteins can be inhibited by a variety of methods, including the use of small molecule inhibitors, proteins, or genetic methods, such as, but not limited to, antisense methods.

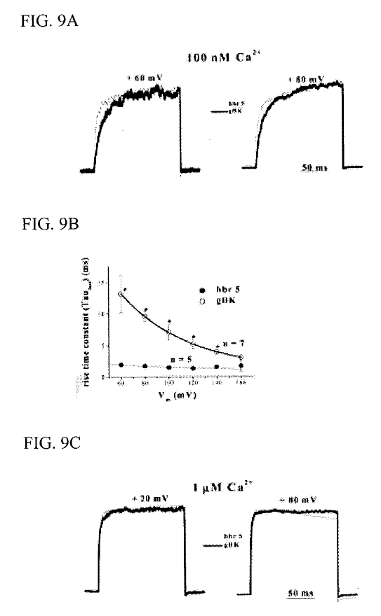

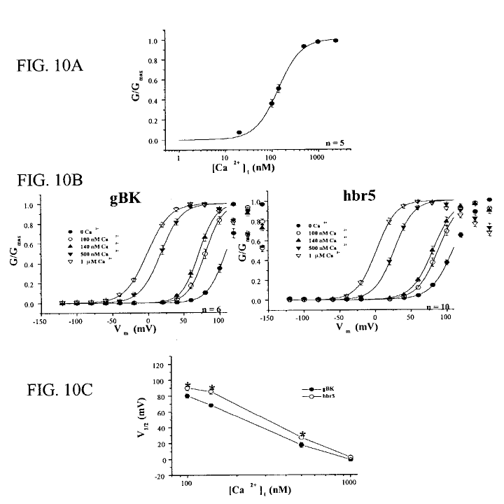

The present disclosure shows that the unique exon at splice site 2 of gBK affects the activation kinetics of the channel as evident by different fast activation time constants (Tau_{fast}) and different slopes of Tau_{fast}-V relations between gBK and hbr5 (FIG. 9). The presence of this exon in gBK also affected the calcium sensitivity, but not the calcium affinity of the channel as gBK and hbr5 have indistinguishable K_{d} values (FIG. 10A), but different V_{1/2} values at the physiological [Ca²⁺]ᵢ range in glioma cells (FIG. 10C). Hence, this data suggests that the unique insert at splice site 2 of gBK affects primarily the gating properties of the channel. It has previously been proposed that the region between S8 and S9 of BK channels, which contains the splice site 2 in gBK, is dispensable. It was thought that the core region of BK channels (S0-S8) determines the voltage dependence of gating, whereas the tail domain (S9-C-terminus) contains two calcium sensing domains, including the “Calcium Bowl”, and an inhibitory domain for voltage-dependent gating. The region between S8 and S9 was considered to be a simple linker to connect these two parts together (Wei et al., 1994; Schreiber and Salkoff, 1997; Schreiber et al., 1999). The present disclosure suggests that this region instead contributes to activation kinetics and Ca²⁺ sensitivity of the gBK channel.

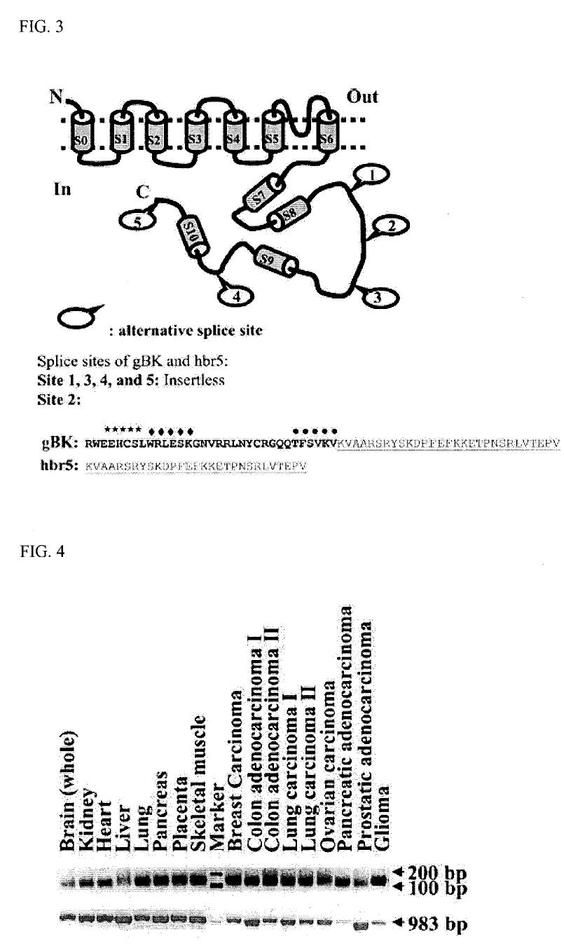

The isolated and purified nucleic acid molecule which encodes the gBK channel is given in SEQ ID NO. 12. The isolated and purified DNA molecule which encodes the novel 99 base pair sequence of gBK at splice site 2 of is given in SEQ ID NO. 13. This sequence lies between the C-terminal hydrophobic domains S8 and S9 (FIG. 3A). The amino acid sequence of novel, human gBK protein is given in SEQ ID NO. 14. The amino acid sequence of the novel 33 amino acid sequence gBK at splice site 2 of the hSlo gene is shown in FIG. 3B and SEQ ID NO 15. The amino acid sequence of SEQ ID NO. 15 is unique and without homology to any reported protein sequence.

As used in this disclosure, an isolated nucleic acid is a nucleic acid the structure of which is not identical to that of any naturally occurring nucleic acid, or to that of any fragment of a naturally occurring genomic nucleic acid spanning more than three separate genes. The term therefore covers, by way of example only, 1) a DNA which has the sequence of part of a naturally occurring genomic DNA molecule, but is not flanked by both of the sequences that flank that part of the molecule in the genome of the organism in which it naturally occurs; 2) a nucleic acid incorporated into a vector or into the genomic DNA of a prokaryote or eukaryote in a manner such that the resulting molecule is not identical to any naturally occurring genomic DNA; 3) a separate molecule such as a cDNA, a genomic fragment, a fragment produced by PCR, or a restriction fragment; and 4) a recombinant nucleotide sequence that is part of a hybrid gene (i.e., a gene encoding a fusion protein).

Nucleic acid encoding for the novel gBK channel, especially those portions of the nucleic acid encoding for the unique splice variant of gBK at splice site 2, may be used to isolate and purify homologues of gBK α-subunit from other organisms, including humans. To accomplish this, gBK channel nucleic acid may be mixed with a sample containing nucleic acids encoding homologues of gBK channels under appropriate hybridization conditions. The hybridized nucleic acid complex may be isolated and the nucleic acid encoding the homologous target may be purified there from. Because the genetic code is degenerate, more than one codon may be used to encode a particular amino acid, and therefore, the amino acid sequence can be encoded by any of a set of similar oligonucleotides. Only one member of the set will be identical to the gBK nucleotide sequence, and will be capable of hybridizing to gBK nucleic acid, under appropriate conditions, even in the presence of oligonucleotides with mismatches. Under alternate conditions, the mismatched oligonucleotides may still hybridize to the gBK channel nucleic acid to permit identification and isolation of gBK channel homologues.

The disclosure also includes nucleic acids that hybridize under stringent conditions (as defined herein) to at least a portion of the nucleotide sequence represented by SEQ ID NOS. 12 or 13, or their complement. The hybridizing portion of the hybridizing nucleic acid is generally 15-50 nucleotides in length. The hybridizing portion of the hybridizing nucleic acid is at least 50% identical to the sequence of at least a portion of the nucleotide sequence represented by SEQ ID NOS. 12 or 13, or its complement. Hybridizing nucleic acids as described herein can be used for many purposes, such as, but not limited to, a cloning probe, an immunological reagent for the production of antibodies, a primer for PCR and other reactions, and a diagnostic probe. Hybridization of the hybridizing nucleic acid is typically performed under stringent conditions. Nucleic acid duplex or hybrid stability is expressed as the melting temperature Tm, which is the temperature at which the hybridizing nucleic acid disassociates with the target nucleic acid. This melting temperature is many times used to define the required stringency conditions. If sequences are to be identified that are related to and/or substantially identical to the nucleic acid sequence represented by SEQ ID NOS. 12 or 13, rather than identical, then it is useful to establish the lowest temperature at which only homologous hybridization occurs with a particular concentration of salt (such as SSC or SSPE).

Assuming that 1% mismatch results in a 1° C. decrease in Tm, the temperature of the final wash in the hybridization reaction is reduced accordingly (for example, if a sequence having a 95% identity with the probe are sought, then the final wash temperature is decreased by 5° C. The change in Tm can be between 0.5° C. and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. The parameters of salt concentration and temperature can be varied to achieve the optimal level of identity between the probe and the target nucleic acid. Additional guidance regarding such conditions is readily available in the art.

As used in this disclosure, the term “percent homology” of two amino acid sequences or of two nucleic acid sequences is determined using the algorithm of Karlin and Altschul (Proc. Natl. Acad. Sci. USA 87: 2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). Blast nucleotide searches are performed with the NBLAST program, score=100, wordlength=12, to obtain nucleotide sequences homologous to a nucleic acid molecule of the invention. Blast protein searches are performed with the XBLAST program, score=50, wordlength=3 to obtain amino acid sequences homologous to a referenced polypeptide. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (XBLAST and NBLAST) are used. See http://www.ncbi.nlm.nih.gov.

The present disclosure also is directed to nucleic acid sequences encoding functional derivatives of gBK. Furthermore, the present disclosure is related to polypeptides encoded by the gBK nucleic acid, or functional derivatives thereof. As used herein, a “functional derivative” includes the “fragments,” “variants,” “degenerate variants,” “mutants” and “chemical derivatives” of the gBK channel. The term “fragment” is meant to refer to any polypeptide subset of gBK polypeptide, or any nucleic acid subset of the gBK nucleic acid sequence. The term “variant” is meant to refer to a polypeptide substantially similar in structure or function to either the gBK polypeptide, or to a fragment thereof and to a nucleic acid sequence substantially similar in structure to the nucleic acid sequence of gBK, or a fragment thereof. The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington The Science and Practice of Pharmacy, 20^{th} Edition.

It is known that there is a substantial amount of redundancy in the codons which code for specific amino acids. Therefore, this disclosure is directed to those nucleic acid sequences which contain alternative codons which code for the eventual translation of the identical amino acid in the gBK channel. For purposes of this specification, a nucleic acid sequence bearing one or more alternative codons will be defined as a “degenerate variation.” For example, conservative amino acid changes, such as, but not limited to, substitution of valine for leucine or asparagine for glutamine may not cause a change in functionality of the polypeptide. Also included within the scope of this disclosure are mutations either in the nucleic acid sequence or the translated protein (“mutants”). The mutants may be isolated from cell lines of tissue or may be introduced via recombinant mechanisms. The mutants may or may not substantially alter the ultimate physical properties of the expressed gBK polypeptide, or functional derivatives thereof. Examples of altered properties of mutants include, but are not limited to changes in the affinity of an enzyme for a substrate or a receptor for a ligand.

gBK channel polypeptides, or functional derivatives thereof, may be expressed either alone or in combination with a polypeptide, nucleic acid, organic or inorganic compound that modulates the function of the gBK channel or its functional derivatives (a “modulating compound”). Expression may be by molecular cloning into an expression vector containing a suitable promoter and other appropriate transcription regulatory elements and transferred into prokaryotic or eukaryotic host cells to produce recombinant molecules. Techniques for such manipulations are within the ordinary skill of one in the art, and representative techniques can be found described in Sambrook, J., et al., Molecular Cloning, Second Edition, 1990, Cold Spring Harbor Press. Expression vectors are defined herein as the nucleic acid sequences that are required for the transcription of cloned copies of nucleic acid and the translation of their mRNAs in an appropriate host. Such vectors can be used to express eukaryotic genes in a variety of hosts such as bacteria, blue green algae, plant cells, insect cells, fungal cells and animal cells. Specifically designed vectors allow the shuttling of nucleic acid between hosts such as bacteria-yeast, or bacteria-animal cells, or bacteria-fungal cells, or bacteria-invertebrate cells. An appropriately constructed expression vector should contain, at the minimum: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. Expression vectors may include, but are not limited to, cloning vectors, modified cloning vectors, specifically designed plasmids or viruses. After expression of the gBK, or functional derivatives thereof, either alone or in combination with a modulating compound, the cell lines expressing these polypeptides may be used to screen for modulators of gBK channel function.

A variety of expression vectors may be used, including, but not limited to, mammalian expression vectors, bacterial expression vectors and insect expression vectors. The expression vectors may be obtained from various commercial suppliers or produced according to specific needs. The choice of the appropriate expression vector is within the ordinary skill of one in the art. The expression vector may contain nucleic acid coding only for the gBK channel, or a function derivative thereof, or may encode for the gBK channel, or a functional derivative thereof, either alone or in combination with a modulating compound.

Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including, but not limited to, Drosophila and silkworm derived cell lines. A variety of cell lines derived from mammalian species which may be suitable for use as host cells are commercially available. The choice of host cells is within the ordinary skill of one in the art.

The expression vectors may be introduced into host cells via any one of a number of techniques including but not limited to transformation, transfection, lipofection, protoplast fusion, and electroporation. The expression vector-containing cells are clonally propagated and individually analyzed to determine whether they produce the compound of interest. Identification of cells expressing the gBK protein, or a functional derivative thereof, can be accomplished by a variety of means, including but not limited to, immunological reactivity, or the presence of host cell-associated gBK channel activity.

Expression of gBK, or functional derivatives thereof, either alone or in combination with a modulating compound may also be performed using in vitro produced synthetic mRNA or isolated native mRNA. Synthetic mRNA or mRNA isolated from gBK channel producing cells can be efficiently translated in various cell-free systems, including but not limited to wheat germ extracts and reticulocyte extracts, as well as efficiently translated in cell based systems, including but not limited to microinjection into frog oocytes.

Following expression of gBK channel, or a functional derivative thereof, in a recombinant host cell, which host cell may also be expressing a modulating compound, such as the β-subunit of a BK channel, or a related K⁺ channel, may be recovered to provide purified gBK protein, or purified gBK protein in association with a modulating compound. Purification methods for isolated expressed protein are well known in the art and within the ordinary skill in the art. Techniques include salt fractionation, ion exchange chromatography, size exclusion chromatography, hydroxylapatite adsorption chromatography, hydrophobic interaction chromatography, immunoaffinity chromatography and affinity chromatography.

Antibodies to desired gBK polypeptides, or functional derivatives thereof, can be produced and used in the methods described herein. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of polypeptides coded for by the gBK nucleic acid, or a functional derivative thereof, in a biological sample, or, alternatively, as a method for the inhibition of abnormal cystin activity. Thus, such antibodies may be utilized as part of treatment methods, and/or may be used as part of diagnostic techniques whereby patients may be tested for the presence of gBK polypeptides, gBK nucleic acids, or a functional derivative of either of the above.

Antibodies to gBK, or a functional derivative thereof, can be purified from mammalian antisera containing antibodies reactive against gBK, or a functional derivative thereof, or are prepared as monoclonal antibodies reactive with gBK or a functional derivative thereof. Specific antibodies are raised by immunizing animals such as mice, rats, guinea pigs, rabbits, goats, horses and the like, with rabbits being preferred, with an appropriate concentration of gBK, or a functional derivative thereof, either with or without an immune adjuvant. Preimmune serum is collected prior to the first immunization to establish a baseline immunoreactivity. Each animal receives between about 0.1 mg and about 1000 mg of gBK, or a functional derivative thereof, either with or without an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of gBK, or a functional derivative thereof, preferably, Freund's complete adjuvant, at multiple sites either subcutaneously (SC), intraperitoneally (IP), or both. Each animal is bled at regular intervals, preferably weekly, to determine antibody titer. The animals may or may not receive booster injections following the initial immunization. Those animals receiving booster injections are generally given an equal amount of the initial antigen in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20 degree C.

Monoclonal antibodies (mAb) reactive with gBK, or a functional derivative thereof, are prepared by immunizing inbred mice, preferably Balb/c, with the appropriate antigen. The mice are immunized by the IP or SC route with about 0.1 mg to about 10 mg, preferably about 1 mg, of antigen in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 0.1 to about 10 mg of antigen in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0, with Sp 2/0 being preferred. The antibody producing cells and myeloma cells are fused in polyethylene glycol, about 1000 molecular weight, at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay using gBK, or a functional derivative thereof (depending on which antigen was used as the antigen for the injections), as the antigen. The culture fluids are also tested in the Ouchterlony precipitation assay to determine the isotype of the mAb. Hybridoma cells from antibody positive wells are cloned by techniques well know in the art.

It is readily apparent to those skilled in the art that the above described methods for producing monospecific antibodies may be utilized to produce antibodies specific for the isolated gBK channel, or functional derivatives thereof, either alone or in combination with a modulating compound.

The antibodies produced above may be used as affinity columns by adding the antibodies to Affigel-10 (Biorad, Hercules, Calif.), according to the manufacturer's directions. After coupling of the antibodies to the column, the column is washed with water followed by 0.23M glycine HCl (pH 2.6) to remove any non-conjugated antibody or extraneous protein. The column is then equilibrated in phosphate buffered saline (pH 7.3) with appropriate detergent and the cell culture supernatants or cell extracts containing gBK, or a functional derivative thereof, wither alone or in combination with a modulating compound, made using appropriate membrane solubilizing detergents are slowly passed through the column. The column is then washed with phosphate buffered saline/detergent until the optical density (A₂₈₀) falls to background, then the protein is eluted with 0.23M glycine-HCl (pH 2.6)/detergent. The purified protein is then dialyzed against phosphate buffered saline/detergent.

The present invention is also directed to methods for screening for compounds which modulate the expression or function of gBK nucleic acid, or a functional derivative thereof, and/or the expression or function of gBK polypeptide, or a functional derivative thereof, in vivo and in vitro, either alone or in combination with a modulating compound. Such an assay may comprise the steps of combining a candidate compound that is suspected to modulate the activity of gBK nucleic acid, or a functional derivative thereof, and/or the expression or function of gBK polypeptide, or a functional derivative thereof, and measuring the effect of the compound on the desired activity.

Compounds may modulate by increasing or attenuating the expression of gBK nucleic acid, or the function of the gBK protein, or a functional derivative thereof, either alone or in combination with a modulating compound. Compounds that modulate the expression or function of nucleic acid encoding gBK, or a functional derivative thereof, and/or the function of gBK polypeptide, or a functional derivative thereof, may be detected by a variety of assays. The assay may be a simple “yes/no” assay to determine whether there is a change in expression or function. The assay may be made quantitative by comparing the expression or function of a test sample with the levels of expression or function in a standard sample. The expression of gBK polypeptide, or a functional derivative thereof, either alone or in combination with a modulating compound, is performed as described above. Such assays are directed to direct inhibitors, as well as indirect inhibitors of this activity.

Kits containing gBK nucleic acids or functional derivatives thereof, or gBK polypeptide, or functional derivatives thereof, antibodies to gBK, and or nucleic acid probes may be prepared. Such kits can be used to detect nucleic acids which hybridize to the gBK nucleic acids, or to nucleic acids coding for functional derivatives of gBK, or to detect the presence of gBK polypeptides, or functional derivatives thereof, in a sample. In addition, such kits would contain the accessory reagents required to complete the assay contemplated by the kit.

Nucleotide sequences complementary to the nucleotide sequences coding for gBK, or a functional derivative thereof, can be synthesized for antisense therapy. These antisense molecules may be DNA, stable derivatives of DNA such as phosphorothioates or methylphosphonates, RNA, stable derivatives of RNA such as 2′-O-alkylRNA, or other antisense oligonucleotide mimetics. These antisense molecules may be introduced into cells by microinjection, liposome encapsulation or by expression from vectors harboring the antisense sequence. Antisense therapy may be particularly useful for the treatment of diseases where it is beneficial to reduce gBK channel activity.

Gene therapy may be used to introduce gBK, or a functional derivative thereof, either alone or in combination with a modulating compound, into the cells of target organs. The gene coding for the appropriate protein can be ligated into viral vectors which mediate transfer of the DNA by infection of recipient host cells. Suitable viral vectors include retrovirus (such as lentiviruses), adenovirus, adeno-associated virus, herpes virus, vaccinia virus, polio virus and the like. Alternatively, DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo, as well as in vivo gene therapy. Gene therapy may be particularly useful for the treatment of diseases where it is beneficial to elevate gBK channel activity.

Pharmaceutically useful compositions comprising gBK nucleic acids or functional derivatives thereof, and their complements and gBK polypeptides or functional derivatives thereof, or functional derivatives thereof, either alone or in combination with modulating compounds, may be formulated according to known methods such as by the admixture of a pharmaceutically acceptable carrier. Examples of such carriers and methods of formulation may be found in Remington The Science and Practice of Pharmacy, 20^{th} Edition. To form a pharmaceutically acceptable composition suitable for effective administration, such compositions will contain an effective amount of the gBK nucleic acids or functional derivatives thereof, their complements, or gBK polypeptides or functional derivatives thereof.

Therapeutic or diagnostic compositions of the invention are administered to an individual in amounts sufficient to treat and/or diagnose disorders related to gBK channel (effective amount). The effective amount may vary according to a variety of factors such as the individual's condition, weight, sex and age. Other factors include the mode of administration. The pharmaceutical compositions may be provided to the individual by a variety of routes such as subcutaneous, topical, oral and intramuscular. Compounds identified according to the methods disclosed herein may be used alone at appropriate dosages defined by routine testing in order to obtain optimal modulation of a gBK channel, or its activity, while minimizing any potential toxicity. In addition, co-administration or sequential administration of other agents may be desirable. The therapeutic or diagnostic agents discussed herein may be used with or without chemical derivatives.

All references to articles, books, patents, websites and other publications and in this disclosure are considered incorporated by reference.

### EXAMPLES

The following examples illustrate the details of the present disclosure only and should not be interpreted to limit the scope of the present disclosure in any way.

### Example 1

### Analysis of BK Channel Expression in Glioma and Non-glioma Tissues

To determine the expression of BK channels in tissues of the brain, the expression of BK channel protein in glioma and non-glioma tissues was examined by immunoblotting. Human biopsy tissues were obtained under an IRB approved protocol from seven patients operated for malignant gliomas. For comparison, tissues of normal cortex without any evident pathology were obtained from two autopsies. The glioma samples examined included two pilocytic astrocytomas (WHO, grade I), two astrocytomas (WHO, grade II), one anaplastic astrocytoma (WHO, grade III), and two GBMs (WHO, grade IV). Tissue samples were prepared by collecting around 0.5 g of tissues and placing the tissue into glass homogenizers with 0.5 ml Homogenization Buffer (HB; 10 mM Tris-HCl, pH7.5, 250 mM Sucrose, 1 mM MgCl₂, 5 mM CaCl₂, and 1 mM PMSF, 10 μg/ml Leupeptin, 1 μg/ml Pepstatin and 1 μg/ml Aproteinin). The tissues were homogenized for 1 min and put on ice. This was repeated for 2 to 3 times and cell debris were spun down and removed at 2,000 g for 5 min at 4° C. Supernatants were collected and proteins were separated by SDS-PAGE and western blot analysis performed.

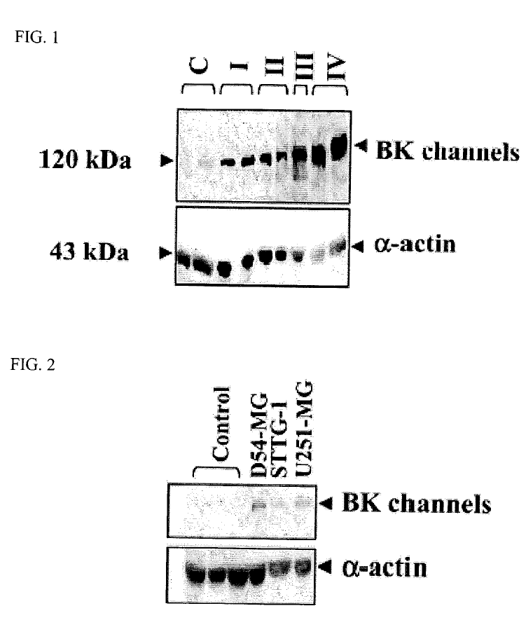

Identical amounts of total protein were loaded (as evident from similar amounts of α-actin used as loading control,) on an 8% SDS-PAGE and probed with polyclonal anti-BK channel antibody, MP-2 (from Dr. Levitan). This antibody recognizes a highly conserved intracellular region at the C-terminus of BK channels from a variety of species including human, rat, and mouse. Considerably higher expression levels of BK channel proteins were observed in all glioma samples compared to the two non-glioma control tissues, despite some visible protein degradation in the glioma samples (FIG. 1). Interestingly, relative BK expression correlated positively with the malignancy grades of the examined tissues. Thus, expression levels in the pilocytic astrocytoma samples, the lowest grade of glioma (WHO I), albeit much more pronounced than in controls were lower than in the samples from astrocytomas or GBM. By far the highest protein level was found in GBM, the most malignant glioma (FIG. 1A, IV).

BK channel expression was also examined in several established and frequently studied cell lines derived from human gliomas by immunoblotting. These cell lines included two GBM derived cell lines (D54-MG and U251-MG, WHO, grade IV), and one astrocytoma derived cell line (STTG-1, WHO, grade III). Normal cortical tissues from three autopsies without evident pathology served control tissue. All three controls showed very low BK protein expression, whereas all three glioma cell lines displayed prominent expression of BK channel protein (FIG. 2). In keeping with the observation from human tissue, BK protein levels correlated positively with enhanced malignancy grades of the cell lines, with significantly higher expression in D54-MG and U251-MG than in STTG-1 cells. These results suggest that relative expression of BK channel protein is notably elevated in human gliomas as compared to non-malignant normal brain.

### Example 2

### Cloning of gBK from Glioma Cells

In order to identify and study the molecular identity of gBK channels, the cDNA encoding this channel from glioma cells was cloned using a reverse transcriptase-polymerase chain reaction (RT-PCR) strategy. Briefly, the 5′ and 3′ cDNA fragments of gBK were first cloned separately. Then the two sequences were subcloned in tandem into the expression vector pcDNA3.1 to yield a 3.5 kb fragment with a 61 bp overlapping sequence, which was eliminated by subsequent mutagenesis. To isolate gBK, a pair of degenerate PCR primers was synthesized (Tseng-Crank et al., 1994). The sequences of the degenerate primers were as follows: s7 forward, 5′GA(A/G)(C/T)TIAA(A/G) (C/TYIGGITT(T/C)ATIGCICA 3′ (SEQ ID NO. 1); s9 reverse, 5′ GGCATIACIA(A/G)(A/G)TTICGIA(A/G) ICCIATIA 3′ (SEQ ID NO. 2). RT-PCR was performed according to the protocol for the RT-PCR kit (Sigma, St. Louis Mo.) from 20 μg glioma cell lineD54-MG whole RNA. The reverse transcription utilized both pd(T)₂₃ and pd(N)₆ primers. The PCR conditions were: 85° C. for 3 min, 94° C. for 1 min, 45° C. for 2 min, and 72° C. for 1 min (30 cycles).

Initial PCR products were cloned into the pSTBlue-1 vector using the pSTBlue-1 Perfectly Blunt Cloning Kit (SOURCE) and sequenced. A 70 bp sequence was identified and matched to the hSlo cDNA sequence. A pair of primers was designed according to this sequence: middle forward, 5′ CCTCTCCACCATGCTTGCCAACCTCTTCTC 3′ (SEQ ID NO. 3); middle reverse, 5′ GGAGAAGAGGTTGGCAAGCATGGTGGAGAG 3′ (SEQ ID NO. 4). The 5′ and 3′ primers of hSlo were designed according to the aligned Slo sequences: forward, 5′ CGGCGGAGGCAGCAGTCTTAGAATGAGTAG 3′ (SEQ ID NO. 5); reverse, 5′GGGGGGACTACAGGGGAAAACAGGGAAAG 3′ (SEQ ID NO. 6). RT-PCR was conducted to clone 5′ and 3′ of hSlo cDNA from D54-MG cells. The products were cloned into the pSTBlue-1 vector and sequenced as above.

The complete gBK cDNA sequence was constructed by assembling the overlapping 5′ and 3′ sequence of gBK cDNA into pcDNA 3.1 in tandem and mutagenesis was conducted to eliminate the overlapping 61 bps between the two fragments. The oligo sequences used for mutagenesis were as follows: forward, 5′ p-CACCATGCTTGCCAACCTCTTCTCCATGAGGTCATTCATAAAGATTGAGG 3′ (SEQ ID NO. 7); reverse, 5′ p-CCTCAATCTTTATGAATGACCTCATGGAGAAGAGGTTGGCAAGCATGGTG 3′ (SEQ ID NO. 8). The programs GENETOOL (BioTools Incorporated, Edmonton, Alberta, Canada), BCM Search Launcher (Baylor College of Medicine, Houston, Tex.) and NCBI BLAST (Bethesda, Md.) were utilized for sequence analysis.

These manipulations generated a full-length CDNA with an open reading frame encoding a protein of 1174 amino acids (SEQ ID NO. 4).

### Example 3

### gBK is Novel BK Channel and the Major BK Channel Isoform in Glioma Cells

Specifically, gBK and hbr5 differ at splice site 2. In hbr5 splice site 2 contains nucleic acid coding for a 29 amino acid insert, while splice site 2 in gBK contains nucleic acid coding for a 62 amino-acid insert composed of an additional 33-amino acid exon adjacent to the N-terminus of the 29 amino acid exon in hbr5 (FIG. 3 and SEQ ID NO. 13). Searching protein databases, it was established that the 33-amino acid insert of gBK is unique and without homology to any reported protein sequence.

To ascertain that gBK is indeed the major BK channel isoform in glioma, PCR primers were synthesized to amplify potential inserts at the splice sites 1, 2, 3 and 4 of BK channels from a CDNA library constructed from human GBM brain tissues. The PCR products from each splicing site were sequenced and analyzed. Splicing site 2 was identified to be the only site of BK channels in glioma cDNA library undergoing alternative splicing and the sequence of the insert was identical to that in gBK. This suggests that gBK is also expressed in acute human glioma and most likely is the only BK channel isoform expressed in glioma.

By applying sequence analysis, the potential phosphorylation sites were identified in the novel gBK exon for various protein kinases, such as Casein Kinase I, Multifunctional calmodulin dependent kinase, and PKC, suggesting that this exon may allow to regulate the biological function of gBK channels in glioma cells.

### Example 4

### Distribution of gBK in Normal and Neoplastic Tissues

PCR was applied to determine the distribution of hSlo alternative splicing variants containing the novel gBK exon among normalized cDNAs from 8 normal and 8 neoplastic tissues. Primers were designed specifically to amplify the novel gBK exon at splicing site 2. PCR primers were designed to amplify specifically the novel gBK exon. The sequences of the primers are as follows: forward, 5′ GTTGGGAAGAACATTGTTCTTTGTGG 3′ (SEQ ID NO. 9), reverse, 5′ATTTAGGTGACACTATAGAAGTGGACTTTGACAGAGAAAGTTTG 3′ (SEQ ID NO. 10). PCR conditions were 95° C. for 30 s, 55° C. for 30 s, 68° C. for 30 s (38 cycles). The primer specificity was determined by sequencing the PCR product from glioma cDNA library with sp6 primer: 5′ ATTTAGGTGACACTATAGAAGTG 3′ (SEQ ID NO. 11).

The sizes and relative quantities of the PCR products amplified from eight normal tissues and eight neoplastic samples are shown in FIG. 4. PCR amplification with the same pair of primers in glioma cDNA was utilized as positive control, amplification from hbr5 vector and without a template were used as negative controls, and expression level of the house-keeping gene G3PDH was used as internal control. All tissues examined showed PCR products with identical size as found in glioma cDNA library, suggesting that hSlo transcripts with this novel exon at splice site 2 are ubiquitously expressed. Note, however, that the relative expression differed substantially as shown in FIG. 4 and the immunoblotting described above (FIGS. 1 and 2), indicating gBK protein levels were elevated in gliomas as compared to normal brain tissues.

### Example 5

### gBK Forms a Functional BK Channel

To test whether the gBK cDNA encodes a functional channel in vivo, gBK expression as a full-length protein in Xenopus oocytes was examined. The hbr5 BK channel was used as a positive control. cRNAs encoding for either gBK or hbr5, respectively, were injected into Xenopus oocytes. Linearized plasmid DNA was transcribed with T7 RNA polymerase in the presence of the cap analog m⁷G(5′)ppp(5′)G with the Ambion mMESSAGE mMACHINE kit (LOCATION). Template DNA was removed using RNase-free DNase I, and the RNA was precipitated with lithium chloride and resuspended in RNA storage buffer (1 mM Sodium Citrate, pH6.4). RNA samples were examined on agarose minigels with ethidium bromide to assure the presence of a single, non-degraded band of the expected size.

Stage VI oocytes from female Xenopus laevis (Xenopus I, Ann Arbor, Mich.) were harvested and incubated at 16° C. before injection. The in vitro transcribed capped cRNA was injected into oocytes with a Nanoject micro-injection system (Drummond Scientific, Broomall, Pa.) at a total volume of about 60 nl (˜100 ng). Oocytes were maintained at 16° C. in sterile oocyte Ringer's incubation solution (OR2) consisting of 92.5 mM NaCl, 2.5 mM KCl, 1 mM MgCl₂, 1 mM Na₂HPO₄, 1 mM CaCl₂ and 5 mM HEPES, 50 U/ml penicillin, and 50 μg/ml streptomycin, pH 7.5. The OR2 solution was changed daily. Functional BK channel expression was observed within 2 days and increasing current levels could be measured up to 4-7 days after injection. Immediately prior to patch-clamp experiments, the vitelline membrane was removed with fine forceps in a hypertonic solution containing 200 mM potassium gluconate, 20 mM KCl, 1 mM MgCl₂, 10 mM EGTA, and 10 mM HEPES (pH adjusted to 7.4 with NaOH).

Three days after injections with gBK or hbr5 cRNA, cell lysates from injected or uninjected oocytes were collected and immunoblotting was performed as described above. As expected, a protein band of approximately 120 kDa was detected in cell lysates from oocytes injected with either gBK or hbr5 cRNA (FIG. 5), demonstrating that both gBK and hbr5 expressed robustly as full-length proteins in Xenopus oocytes. Endogenous BK channel expression could not be detected in uninjected oocytes with the MP2 antibody.

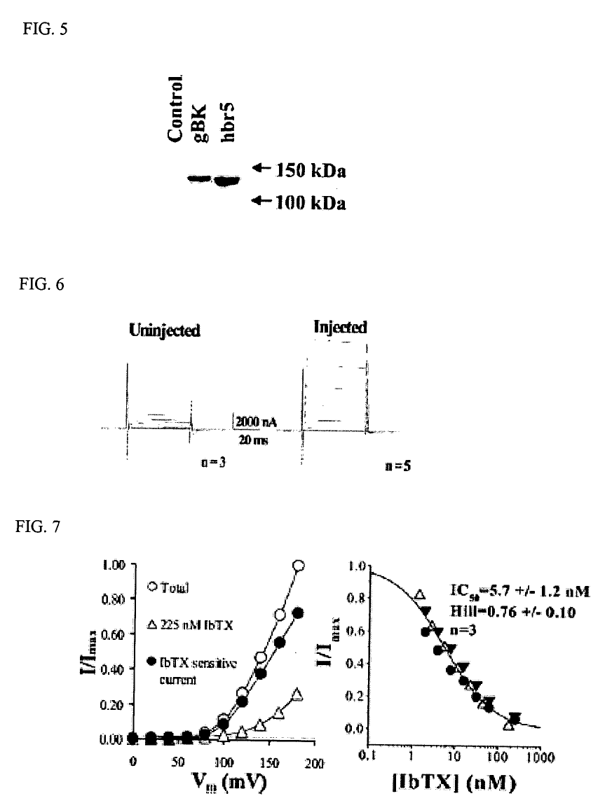

The ability of gBK protein expressed in Xenopus oocytes to form functional BK channels was examined by two-electrode voltage-clamp. Two days after cRNA injection, the oocytes were placed in a 100 μl chamber with continuous perfusion of OR2 solution. The oocytes were voltage-clamped at −20 mV and then jumped to 10 voltage steps in 20 mV increments (0˜+180 mV) using a GeneClamp 500 amplifier (Axon Instruments, Foster City, Calif.). The current signal was low-pass-filtered at 2 kHz and digitized at 10 kHz. Data were collected via a Power Macintosh 7300 computer (Apple, Cupertino, Calif.) running Igor Pro software (WaveMetrics, Lake Oswego, Oreg.) with Pulse Control macro (Instrutech, Port Washington, N.Y.). For IbTX blockage experiments, IbTX was applied at increasing concentrations by switching from control solution to each IBTX solution, and voltage steps were applied after constant perfusion of an IbTX solution until it reached steady-state, which, for low concentrations of IbTX, took up to 20 min. Two days after cRNA injection, large amplitude voltage-activated outward currents were recorded (FIG. 6, right panel), which were not detected in uninjected oocytes (FIG. 6, left panel). To confirm that the outward currents were mediated by BK channels, we applied the highly selective BK channel blocker IbTX. Greater than 60% of total currents were inhibited by 100 nM IbTX. The same concentration did not affect currents in uninjected oocytes. Thus, gBK cDNA encodes a functional BK channel in Xenopus oocytes.

A complete dose-response curve for inhibition of gBK currents by IbTX was established from three oocytes by voltage-clamp recordings. FIG. 7 (left panel) shows the normalized current (I/Iₘₐₓ)-voltage relations before (Total, open circles), and after addition of 225 nM IbTX (225 nM IbTX, open triangles), and the IbTX sensitive currents (closed circles) that were obtained by subtracting 225 nM IbTX currents from total currents. FIG. 7 (right panel) shows pooled IbTX dose-responses from three oocytes (three symbols represent data from three oocytes respectively). The IC₅₀ and Hill coefficient of IbTX inhibition of gBK currents were determined by least-squares fit of the I/Iₘₐₓ-[IbTX] relation of IbTX sensitive currents to a Hill equation. This yielded an IC₅₀ of 5.7±1.23 nM and a Hill coefficient of 0.76±0.10 (±S.D., n=3) for IbTX block of gBK currents. These values are similar to those obtained in D54-MG glioma cells (Ransom and Sontheimer, 2001), suggesting that gBK may encode the endogenous BK currents in glioma cells.

### Example 6

### Single-channel Conductance of gBK in Xenopus Oocytes

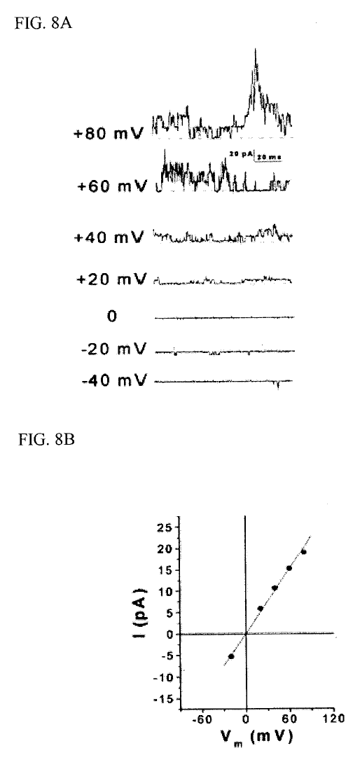

As discussed above, different splice variants of BK channels can differ in single-channel conductance, kinetics of activation, and calcium sensitivity (Lagrutta et al., 1994; Saito et al., 1997; Xie and McCobb, 1998; Ramanathan et al., 1999). The single-channel conductance of gBK was examined from inside-out patches. FIG. 8A illustrates representative recordings of gBK unitary currents in symmetrical solutions containing 145 mM K-gluconate with 100 nM free [Ca²⁺]ᵢ at different holding potentials. The unitary currents of gBK channel were determined from Gaussian fits of amplitude frequency histograms using Fetchan and Pstat (Axon Instrument), and the single-channel I-V relation of gBK (filled circle) is plotted in FIG. 8B. The slope of the I-V relation suggests a single-channel conductance of 250 pS (±10.7 pS, n=5) for gBK, which is consistent with the unitary conductance of endogenous BK currents (250-300 pS) determined in patch recordings from five different glioma cell lines with symmetric 150-160 mM K+-solution (Brismar and Collins, 1989).

The unitary conductance of hbr5 was determined to be 269 pS (±10.8 pS, n=6) and was thus essentially identical to that of gBK, suggesting that the splice insert in gBK does not affect the unitary conductance.

### Example 7

### gBK Shows Altered Activation Kinetics

The activation kinetics of gBK in response to depolarizing voltage steps using inside-out macropatches was also examined (FIGS. 9A and C). Mean values were derived by fitting the data to a double exponential function (FIG. 9B). Interestingly, the fast rise time constants (Tau_{fast})-V relations differed significantly between gBK and hbr5 at voltages more negative than 140 mV (p<0.05) (FIG. 9B), whereas their slow rise time constants (Tau_{slow})-V relations were indistinguishable. These values were obtained at 100 nM free [Ca²⁺]ᵢ, a value that is within the range of the resting [Ca²⁺]ᵢ levels in glioma cells. However, at 1 μM free [Ca²⁺]ᵢ, both Tau_{fast}-V and Tau_{slow}-V relations of gBK and hbr5 were essentially identical (data not shown), and normalized traces at two voltage steps are shown in FIG. 9C. Furthermore, the experimentally observed activation time constant determined at 100 mV and 10 ms after onset of the voltage step was 12 ms (±1.3 ms, n=7), and identical to the value reported for native BK currents in glioma cells (Ransom and Sontheimer, 2001), again suggesting the gBK encodes predominant BK currents in glioma cells.

All macropatch experiments were performed using the gigohm seal patch-clamp method in the excised inside-out configuration (Hamill et al., 1981). Patch pipettes were pulled from thin-walled borosilicate glass (TW150F-40, WPI, Sarasota, Fla.) on a PP-830 puller (Narishige Instruments, Japan) and flame-polished on a microforge (MF-83, Narishige Instruments) and had resistances of 4-7 MΩ. Macropatch pipettes were pulled with very steep taper, which resulted in the excision of a large area of membrane due to the propensity of oocyte membranes to form seals as far as 20-100 μm into the electrode (Ruknudin et al., 1991). The macropatch currents were amplified using an Axopatch-1D amplifier (Axon Instruments) controlled by a PC-compatible microcomputer (Dell Computers, Dallas, Tex.) running pClamp8 (Axon Instrument). Data was stored directly to disk using a Digidata 1200 A-D interface (Axon Instruments) and acquired at 10 kHz and filtered at 2 kHz. Capacitance compensation was performed using the built-in amplifier circuitry. No series resistance compensation was used, and leak currents were not subtracted from macropatch currents. Pipette potentials were nulled immediately prior to seal formation.

During seal formation, oocytes were bathed in ND96 (96 mM NaCl, 2 mM KCl, 1 mM MgCl₂, and 5 mM HEPES, pH 7.5 supplemented with 2.5 mM sodium pyruvate). After excision, patches were quickly moved into a flowing zero Ca²⁺ solution. For inside-out recordings, the pipette extracellular solution was 145 mM K⁺-gluconate, 5 mM KCl, 2.5 mM MgCl₂, 10 mM HEPES and 1 mM EGTA (pH adjusted to 7.4 with KOH). The intracelluar (bath) solution was 145 mM K⁺-gluconate, 5 mM KCl, 2.5 mM MgCl₂, 10 mM HEPES and 1 mM EGTA (pH adjusted to 7.2 with KOH). Recording solutions contained gluconate as a nonpermeant anion to prevent the activation of calcium-activated chloride channels endogenous to oocytes (Miledi, 1982). Sufficient CaCl₂ was added to obtain the desired free [Ca²⁺]. The calcium to add to our intracellular solutions in experiments with elevated free calcium concentrations was calculated with a software program based on equations provided in Marks and Maxfield (Marks and Maxfield, 1991). This program takes into account pH and the type of chelators present. Corrections were made for EGTA purity. For target free Ca²⁺ concentrations of 0.1, 0.14, 0.5 and 1 μM, 0.387, 0.470, 0.746 and 0.844 mM Ca²⁺, respectively, was added.

### Example 8

### Calcium Sensitivity of gBK Expressed in Xenopus Oocytes

To measure the calcium sensitivity of the BK channel currents recorded in oocyte macro-patches, oocytes expressing gBK and hbr5 were examined separately. Of the cloned BK channels, hbr5 represents one of the most Ca²⁺ sensitive BK channel isoforms. FIG. 10A summarizes the steady—state Ca²⁺—dependence curves of gBK derived from five patches at +80 mV testing potential. To obtain these curves, conductance-[Ca²⁺]ᵢ relations from each patch were plotted and fitted to a Hill equation. With the Gₘₐₓ values obtained from the fit, the conductances were normalized and averaged. Normalized conductances (G/Gₘₐₓs, close circles), were then plotted against [Ca²⁺]ᵢ and fitted to a Hill equation (solid curve). The normalized conductance of gBK reached the plateau at around 1 μM [Ca²⁺]ᵢ with an apparent Kd of 137 nM (±22.3 nM, n=5), indicating overall high Ca²⁺ sensitivity of gBK channel. With the same strategy, the apparent Kd of hbr5 was obtained (150 nM±20 nM, n=6, data not shown). This suggests that the apparent Kds of gBK and hbr5 are indistinguishable.

We further examined the steady state G/Gₘₐₓ-voltage relations of gBK. FIG. 10B summarizes recordings obtained from gBK (Left panel) and hbr5 expressed oocytes (Right panel) at different Ca²⁺ concentrations, respectively. Steady-state G/Gₘₐₓ values were derived from conductance-voltage relations of individual patches that were normalized and fitted to a Boltzmann equation. Averaged G/Gₘₐₓ values from six patches for gBK and ten patches for hbr5 were plotted against testing potentials in FIG. 9B. As previously reported, increasing intracellular Ca²⁺ shifted the conductance-voltage curve to the left for both gBK and hbr5, and very depolarized voltage (>+100 mV) were required to significantly activate these channels at zero Ca²⁺.

The V_{1/2} values, the voltages at which 50% of channels are active, is a convenient means to compare the calcium sensitivity of BK channels. The V_{1/2} values in the physiological range of Ca²⁺ concentrations were determined and plotted against free [Ca²⁺]ᵢ in FIG. 10C. Asterisks in FIG. 10C indicate that the differences between the two isoforms at same Ca²⁺ concentrations were significantly different (p<0.05) based on one-way ANOVA analysis. Our data suggests that over a physiological relevant [Ca 2+]ᵢ range in glioma cells (100 nM-500 nM), the Ca²⁺ sensitivity of gBK is significantly higher than previously identified BK channels in human preparations (Tseng-Crank et al., 1994; DeCoursey et al., 1996; Hurley et al., 1999). Moreover, the Ca²⁺ sensitivity of gBK was identical to the native BK currents that have been reported recently in glioma cells (Ransom and Sontheimer, 2001).

### Example 9

### Ca²⁺ Sensitivity of gBK Channel Currents Isolated from STTG-1 and D54MG Cells

Outside-out patches were isolated from STTG-1 cells and the voltage dependence of gBK currents were isolated with different [Ca²⁺]ᵢ. Currents were integrated over the period of a voltage step and the average conductance was calculated from these areas (Ransom and Sontheimer, 2002). For each patch, 5-20 families of current traces over a range of voltages were obtained and the average conductance values at each membrane potential were determined. The normalized conductance was plotted as a function of membrane potential (activation curves) and fit to the Boltzman equation to obtain half-maximal voltages, V_{0.5}. A total of 45 outside-out patches and their corresponding activation curves with [Ca²⁺]i of 0 (10 mM EGTA added), 1.5×10−7 M and 2.1×10−6 M were determined. These calcium concentrations resulted in V_{0.5} values of +144 mV, +79 mV and −16 mV, respectively. (FIGS. 11A and 11B). For comparison, the same set of experiments was performed on the hbr5 BK channel (Ransom and Sontheimer, 2002). V_{0.5} for hbr5 BK channel at [Ca²⁺]i of 0 (10 mM EGTA added), 1.5×10−7 M and 2.1×10−6 M were +118 mV, +52 mV and −23 mV (FIGS. 12A and 12B). Therefore, as compared with expression studies of gBK and hbr5 in oocytes (where modest differences in calcium sensitivities were found), the results in glioma cell lines suggest that gBK channels are much more sensitive than hbr5. One potential explanation for these results could be the association of a β-subunit associating with gBK channels to modify the calcium sensitivity.

### Example 10

### gBK Channels are Sensitive to Tetrandrine

If gBK channels are associated with β-subunits, then they should be sensitive to tetrandrine, a plant alkaloid. 3 uM tetrandrine has been reported to inhibit β-subunit-containing channels over 50%. 3 uM tetrandrine resulted in 63+/−8% inhibition of gBK currents in outside-out patches (FIG. 13A). This same concentration of tetrandrine resulted only 14% inhibition of hbr5 currents (FIG. 13B). The results of application of 3 uM and 30 uM tetrandrine to gBK and hbr5 currents in outside-out patches are shown in FIG. 13C. These results are consistent with β-subunit expression by glioma cell lines, but not by HEK cells transfected with hbr5.

Data Analysis

Data were analyzed off-line with the software packages Clampfit8 (Axon Instruments), Origin (v.6.0, MicroCal Software, Northhampton, Mass.) and Excel 2000 (Microsoft, Seattle, Wash.).

Dose-response curves for IbTX were constructed first by measuring the leak-subtracted steady-state currents at each IbTX concentration at +160 mV. Data from each oocyte was fitted to a modified Hill equation: I=Iₘₐₓ/(1+([IbTX]/IC₅₀)ⁿ), where I is the IbTX sensitive current, Iₘₐₓ is the maximum current of the fit, IC₅₀ is the half-maximal inhibitory concentration of IbTX, and n is the Hill coefficient. The IbTX sensitive currents were normalized to the maximum value determined from the fit and normalized currents from each oocyte were pooled and plotted against the applied IbTX concentration. IC₅₀ and Hill coefficient were obtained from averaged data from three oocytes.

Calcium dependence curves were constructed similarly as the IbTX dose-response curve except data were fitted to the following equation: G=Gₘₐₓ/(1+(K_{d}/[Ca]ᵢ)ⁿ, where Gₘₐₓ is the maximal conductance, K_{d} is the apparent Ca²⁺ dissociation constant, and n is the Hill coefficient. The curve was obtained from averages of normalized data from five micropatches. The normalized conductance (G/Gₘₐₓ) curves were obtained by first measuring the steady state currents for each macropatch. Since we used symmetrical K⁺ and reversal potential was zero, we calculated the conductance for each test potential: G =I/(Vₘ−0). These data were plotted against test voltages, fitted to the Boltzmann equation: G=Gₘₐₓ/(1+e^{-(V-V1/2)zF/RT}) (Weiss and Magleby, 1990), and then normalized to the maximum value obtained from the fit. The resulting G/Gₘₐₓ, in turn, were averaged, plotted against test voltages and fitted to the Boltzmann equation, also.

### REFERENCES

Adelman J P, Shen K Z, Kavanaugh M P, Warren R A, Wu Y N, Lagrutta A, Bond C T, North R A (1992) Calcium-activated potassium channels expressed from cloned complementary DNAs. Neuron 9: 209-216.

Atkinson N S, Robertson G A, Ganetzky B (1991) A component of calcium-activated potassium channels encoded by the Drosophila slo locus. Science 253: 551-555.

Brenner R, Perez G J, Bonev A D, Eckman D M, Kosek J C, Wiler S W, Patterson A J, Nelson M T, Aldrich R W (2000) Vasoregulation by the betal subunit of the calcium-activated potassium channel [In Process Citation]. Nature 407: 870-876.

Bringmann A, Faude F, Reichenbach A (1997) Mammalian retinal glial (Muller) cells express large-conductance Ca(2+)-activated K+ channels that are modulated by Mg2+ and pH and activated by protein kinase A. Glia 19: 311-323.

Bringmann A, Francke M, Pannicke T, Biedermann B, Faude F, Enzmann V, Wiedemann P, Reichelt W, Reichenbach A (1999) Human Muller glial cells: altered potassium channel activity in proliferative vitreoretinopathy. Invest Ophthalmol Vis Sci 40: 3316-3323.

Bringmann A, Francke M, Pannicke T, Biedermann B, Kodal H, Faude F, Reichelt W, Reichenbach A (2000) Role of glial K(+) channels in ontogeny and gliosis: a hypothesis based upon studies on Muller cells. Glia 29: 35-44.

Bringmann A, Reichenbach A (1997) Heterogenous expression of Ca2+-dependent K+ currents by Muller glial cells. Neuroreport 8: 3841-3845.

Brismar T, Collins V P (1989) Potassium and sodium channels in human malignant glioma cells. Brain Res 480: 259-267.

Butler A, Tsunoda S, McCobb D P, Wei A, Salkoff L (1993) mSlo, a complex mouse gene encoding “maxi” calcium-activated potassium channels. Science 261: 221-224.

Christensen O, Zeuthen T (1987) Maxi K+ channels in leaky epithelia are regulated by intracellular Ca2+, pH and membrane potential. Pflugers Arch 408: 249-259.

Collins V P (1994) Epidermal growth factor receptor gene and its transcripts in glioblastomas. Recent Results Cancer Res 135: 17-24.

DeCoursey T E, Kim S Y, Silver M R, Quandt F N (1996) Ion channel expression in PMA-differentiated human THP-1 macrophages. J Membr Biol 152: 141-157.

Ding H, Roncari L, Shannon P, Wu X, Lau N, Karaskova J, Gutmann D H, Squire J A, Nagy A, Guha A (2001) Astrocyte-specific expression of activated p21-ras results in malignant astrocytoma formation in a transgenic mouse model of human gliomas. Cancer Res 61: 3826-3836.

Ferrer J, Wasson J, Salkoff L, Permutt M A (1996) Cloning of human pancreatic islet large conductance Ca(2+)-activated K+ channel (hSlo) cDNAs: evidence for high levels of expression in pancreatic islets and identification of a flanking genetic marker. Diabetologia 39: 891-898.

Gallin E K (1984) Calcium- and voltage-activated potassium channels in human macrophages. Biophys J 46: 821-825.

Gitter A H, Beyenbach K W, Christine C W, Gross P, Minuth W W, Fromter E (1987) High-conductance K+ channel in apical membranes of principal cells cultured from rabbit renal cortical collecting duct anlagen. Pflugers Arch 408: 282-290.

Golding N L, Jung H Y, Mickus T, Spruston N (1999) Dendritic calcium spike initiation and repolarization are controlled by distinct potassium channel subtypes in CA1 pyramidal neurons. J Neurosci 19: 8789-8798.

Hanaoka K, Wright J M, Cheglakov I B, Morita T, Guggino W B (1999) A 59 amino acid insertion increases Ca(2+) sensitivity of rbslo1, a Ca2+-activated K(+) channel in renal epithelia. J Membr Biol 172: 193-201.

Hernandez M, Barrero M J, Crespo M S, Nieto M L (2000) Lysophosphatidic acid inhibits Ca2+ signaling in response to epidermal growth factor receptor stimulation in human astrocytoma cells by a mechanism involving phospholipase C(gamma) and a G(alphai) protein. J Neurochem 75: 1575-1582.

Huang Y, Rane S G (1994) Potassium channel induction by the Ras/Raf signal transduction cascade. J Biol Chem 269: 31183-31189.

Hurley B R, Preiksaitis H G, Sims S M (1999) Characterization and regulation of Ca2+-dependent K+ channels in human esophageal smooth muscle. Am J Physiol 276: G843-52.

Jones E M, Gray-Keller M, Art J J, Fettiplace R (1999) The functional role of alternative splicing of Ca(2+)-activated K+ channels in auditory hair cells. Ann NY Acad Sci 868: 379-385.

Kamouchi M, Trouet D, De Greef C, Droogmans G, Eggermont J, Nilius B (1997) Functional effects of expression of hslo Ca2+ activated K+ channels in cultured macrovascular endothelial cells. Cell Calcium 22: 497-506.

Kodal H, Weick M, Moll V, Biedermann B, Reichenbach A, Bringmann A (2000) Involvement of calcium-activated potassium channels in the regulation of DNA synthesis in cultured Muller glial cells. Invest Ophthalmol Vis Sci 41: 4262-4267.

Lagrutta A, Shen K Z, North R A, Adelman J P (1994) Functional differences among alternatively spliced variants of Slowpoke, a Drosophila calcium-activated potassium channel. J Biol Chem 269: 20347-20351.

Lerche H, Fahlke C, Iaizzo P A, Lehmann-Horn F (1995) Characterization of the high-conductance Ca(2+)-activated K+ channel in adult human skeletal muscle. Pflugers Arch 429: 738-747.

Lingle C J, Solaro C R, Prakriya M, Ding J P (1996) Calcium-activated potassium channels in adrenal chromaffin cells. Ion Channels 4:261-301: 261-301.

Liu, X, Reinhart, P H, Sontheimer, H (2002) Cloning and characterization of glioma BK, a novel BK channel isoform highly expressed in human glioma cells J of Neuroscience, In Press.

MacDermott A B, Weight F F (1982) Action potential repolarization may involve a transient, Ca2+-sensitive outward current in a vertebrate neurone. Nature 300: 185-188.

Marks P W, Maxfield F R (1991) Preparation of solutions with free calcium concentration in the nanomolar range using 1,2-bis(o-aminophenoxy)ethane-N,N,N′,N′-tetraacetic acid. Anal Biochem 193: 61-71.

Marty A (1989) The physiological role of calcium-dependent channels. Trends Neurosci 12: 420-424.

McCobb D P, Fowler N L, Featherstone T, Lingle C J, Saito M, Krause J E, Salkoff L (1995) A human calcium-activated potassium channel gene expressed in vascular smooth muscle. Am J Physiol 269: H767-77.

McManus O B (1991) Calcium-activated potassium channels: regulation by calcium. J Bioenerg Biomembr 23: 537-560.

Merzak A, Pilkington G J (1997) Molecular and cellular pathology of intrinsic brain tumours. Cancer Metastasis Rev 16: 155-177.

Miledi R (1982) A calcium-dependent transient outward current in Xenopus laevis oocytes. Proc R Soc Lond B Biol Sci 215: 491-497.

Nelson M T, Quayle J M (1995) Physiological roles and properties of potassium channels in arterial smooth muscle. Am J Physiol 268: C799-C822.

Newman E A (1985) Voltage dependent calcium and potassium channels in retinal glial cells. Nature 317: 809-811.

Nowak L, Ascher P, Berwald-Netter Y (1987) Ionic channels in mouse astrocytes in culture. J Neurosci 7: 101-109.

Poolos N P, Johnston D (1999) Calcium-activated potassium conductances contribute to action potential repolarization at the soma but not the dendrites of hippocampal CA1 pyramidal neurons. J Neurosci 19: 5205-5212.

Puro D G, Roberge F, Chan C C (1989) Retinal glial cell proliferation and ion channels: a possible link. Invest Ophthalmol Vis Sci 30: 521-529.

Ramanathan K, Michael T H, Jiang G J, Hiel H, Fuchs P A (1999) A molecular mechanism for electrical tuning of cochlear hair cells. Science 283: 215-217.

Ransom C B, Liu, X., Sontheimer, H (2002) BK channels in human glioma cells have enhanced calcium sensitivity. Glia, In Press.

Ransom C B, Sontheimer H (2001) BK Channels in Human Glioma Cells. J Neurophysiol 85: 790-803.

Repp H, Draheim H, Ruland J, Seidel G, Beise J, Presek P, Dreyer F (1993) Profound differences in potassium current properties of normal and Rous sarcoma virus-transformed chicken embryo fibroblasts. Proc Natl Acad Sci USA 90: 3403-3407.

Repp H, Matzek A, Draheim H, Malettke N, Dreyer F (1995) Epidermal Growth Factor, Platelet-derived Growth Factor AB, Insulin, Lysophosphatidic Acid, and Serum Modulate K+ Channel Properties in Chicken Embryo Fibroblasts. Cell Physilo Biochem 5: 145-154.

Robitaille R, Charlton M P (1992) Presynaptic calcium signals and transmitter release are modulated by calcium-activated potassium channels. J Neurosci 12: 297-305.

Robitaille R, Garcia M L, Kaczorowski G J, Charlton M P (1993) Functional colocalization of calcium and calcium-gated potassium channels in control of transmitter release. Neuron 11: 645-655.

Saito M, Nelson C, Salkoff L, Lingle C J (1997) A cysteine-rich domain defined by a novel exon in a slo variant in rat adrenal chromaffin cells and PC12 cells. J Biol Chem 272: 11710-11717.

Schreiber M, Salkoff L (1997) A novel calcium-sensing domain in the BK channel. Biophys J 73: 1355-1363.

Schreiber M, Yuan A, Salkoff L (1999) Transplantable sites confer calcium sensitivity to BK channels. Nat Neurosci 2: 416-421.

Soroceanu L, Manning T J, Jr., Sontheimer H (1999) Modulation of glioma cell migration and invasion using Cl(−) and K(+) ion channel blockers. J Neurosci 19: 5942-5954.

Tian L, Duncan R R, Hammond M S, Coghill L S, Wen H, Rusinova R, Clark A G, Levitan I B, Shipston M J (2001) Alternative Splicing Switches Potassium Channel Sensitivity to Protein Phosphorylation. J Biol Chem 276: 7717-7720.

Tseng-Crank J, Foster C D, Krause J D, Mertz R, Godinot N, DiChiara T J, Reinhart P H (1994) Cloning, expression, and distribution of functionally distinct Ca(2+)-activated K+ channel isoforms from human brain. Neuron 13: 1315-1330.

Turnheim K, Costantin J, Chan S, Schultz S G (1989) Reconstitution of a calcium-activated potassium channel in basolateral membranes of rabbit colonocytes into planar lipid bilayers. J Membr Biol 112: 247-254.

Wei A, Solaro C, Lingle C, Salkoff L (1994) Calcium sensitivity of BK-type KCa channels determined by a separable domain. Neuron 13: 671-681.

White T W, Bruzzone R, Wolfram S, Paul D L, Goodenough D A (1994) Selective interactions among the multiple connexin proteins expressed in the vertebrate lens: the second extracellular domain is a determinant of compatibility between connexins. J Cell Biol 125: 879-892.

Whitfield J F, Bird R P, Chakravarthy B R, Isaacs R J, Morley P (1995) Calcium-cell cycle regulator, differentiator, killer, chemopreventor, and maybe, tumor promoter. J Cell Biochem Suppl 22: 74-91.

Wiecha J, Munz B, Wu Y, Noll T, Tillmanns H, Waldecker B (1998) Blockade of Ca2+ activated K+ channels inhibits proliferation of human endothelial cells induced by basic fibroblast growth factor. J Vasc Res 35: 363-371.

Xie J, McCobb D P (1998) Control of alternative splicing of potassium channels by stress hormones. Science 280: 443-446.

Zahradnikova A, Zahradnik I (1992) Single potassium channels of human glioma cells. Physiol Res 41: 299-305.

What is claimed:

1. An isolated and purified nucleic acid molecule comprising the nucleotide sequence of SEQ ID NO: 12, or a functional derivative of SEQ ID. NO: 12.

2. The nucleic acid molecule of claim 1 where said sequence encodes a polypeptide having the amino acid sequence of SEQ ID NO: 14.

3. The nucleic acid molecule of claim 1 where said sequence encodes a polypeptide having the amino acid sequence of SEQ ID NO: 14, or a fragment of SEQ ID NO: 14 at least 5 residues in length.

4. An isolated and purified nucleic acid molecule comprising the nucleotide sequence of SEQ ID NO: 13, or a functional derivative of SEQ ID. NO: 13.

5. The nucleic acid molecule of claim 4 where said sequence encodes a polypeptide having the amino acid sequence of SEQ ID NO: 15.

6. The nucleic acid molecule of claim 4 where said sequence encodes a polypeptide having the amino acid sequence of SEQ ID NO: 15, or a fragment of SEQ ID NO: 15 at least 5 residues in length.

7. The nucleic acid molecule of claim 4 comprising a sequence of which is at least 50% identical to SEQ ID NO: 13

8. An isolated nucleic acid comprising a sequence that hybridizes under highly stringent conditions to a hybridization probe the nucleotide sequence of which consists of the sequences selected from the group consisting of SEQ ID NO: 12, the complement of SEQ ID NO: 12, a fragment of SEQ ID NO: 12 at least 15 nucleotides in length, the complement of a fragment of SEQ ID NO: 12 at least 15 nucleotides in length SEQ ID NO: 13, the complement of SEQ ID NO: 13, a fragment of SEQ ID NO: 13 at least 15 nucleotides in length and the complement of a fragment of SEQ ID NO: 13 at least 15 nucleotides in length.

9. A purified polypeptide the amino acid sequence of which comprises SEQ ID NO: 14, or a degenerate variant of SEQ ID NO: 14.

10. The polypeptide of claim 9 where the V_{0.5} value at an intracellular calcium concentration of 0 M is +144 mV when said polypeptide is expressed in STTG-1 cells.

11. The purified polypeptide of claim 9 where said amino acid sequence comprises at least 5 consecutive amino acids of SEQ ID NO: 14.

12. A purified polypeptide the amino acid sequence of which comprises SEQ ID NO: 15, or a degenerate variant of SEQ ID NO: 15.

13. The purified polypeptide of claim 12 where said amino acid sequence comprises at least 5 consecutive amino acids of SEQ ID NO: 15.

14. The purified polypeptide of claim 12 where said amino acid sequence is at least 50% identical to SEQ ID NO: 15.

15. The purified polypeptide of claim 12 further comprising phosphorylation sites for at least one protein kinase.

16. The purified protein of claim 15 where the at least one protein kinase is selected from the group consisting of casein kinase I, protein kinase C and multi functional calmodulin-dependent kinase.

17. An expression vector comprising the nucleic acid of claim 3 operably linked to an expression control sequence.

18. A non-human cell comprising the nucleic acid of claim 3 operably linked to an expression control sequence.

19. The cell of claim 18 further comprising a modulating compound.

20. The cell of claim 19 where the modulating compound is a β-subunit of a BK channel.

21. A method of producing a polypeptide, the method comprising culturing the cells of claim 18 under conditions permitting expression of the polypeptide.

22. The method of claim 21 further comprising purifying the polypeptide from the cell of the medium of the cell.
